(19) United States
(12) Patent Application Publication
(10) Pub. No.: US 2015/0359667 A1
(43) Pub. Date: Dec. 17, 2015

(54) SYSTEM FOR EYE MEDICATION COMPLIANCE AND TRACKING

(71) Applicant: Vesta Brue, Lexington, KY (US)

(72) Inventor: Vesta Brue, Lexington, KY (US)

(21) Appl. No.: 14/305,631

(22) Filed: Jun. 16, 2014

(51) Int. Cl. A61F 9/00; H04L 29/08

(52) U.S. Cl. CPC A61F 9/0008; H04L 67/02; H04L 67/10

(57) ABSTRACT

Disclosed herein are a dispenser that contains a vial of eyedrops and a corresponding base station, both of which are designed according to medication dosing alerts, poka-yoke principles, and audible instructions to reduce the chances of improper usage. An example base station includes a set of dispenser receptacles, each receptacle being configured to receive and contain a respective specifically shaped dispenser from a set of dispensers associated with the base station, wherein each of the set of dispensers is uniquely shaped. The example base station includes a communications interface configured to receive eyedrop usage data from at least one of the set of dispensers, a storage device configured to store the eyedrop usage data, and an external communications interface configured to transmit the eyedrop usage data to at least one external device. The dispensers can hold a vial of medicine, and can include control circuits, sensors, buttons, a notification module, and communications interfaces.

### BACKGROUND

1. Technical Field

The present disclosure relates to administering eyedrops and more specifically to accurately tracking and reporting patient eye drop usage in a way that is simple for patients and accurate for care providers.

2. Introduction

Modern medical practice includes various treatments for eye conditions such as glaucoma, pink eye, bacterial conjunctivitis, viral conjunctivitis, allergic conjunctivitis, dry eyes, swelling, and so forth. Often, these treatments involve a regimen of eyedrops administered at regular intervals over a period of time. For example, a doctor may prescribe the medicine Zaditor with instructions for the patient to administer 1 drop to each eye (and more that 1 drop if so prescribed) every 4 hours for one week. However, treatment can be hindered as patients fail to follow the prescribed eyedrop regime. Additionally, patients using such eye drops often have vision impairment, and have significant difficulty in locating and identifying the proper eyedrop containers, as well as reading the associated directions. These problems are exacerbated when a doctor prescribes a patient multiple eyedrops to be applied at intervals such that a drop does not wash out a prior eyedrop that was applied. Maintaining timely separations between drops is critical to successful performance of some drugs. Then, a vision impaired patient struggles to differentiate eye-drop containers from each other, struggles to remember to apply the drops at the appropriate intervals, struggles to read the instructions on the eye-drop bottle for how many drops to apply, and so forth. Further, a doctor or primary caregiver not in residence has no way to conveniently and accurately follow up to verify that the treatment regime with the eyedrops is being followed. Each of these difficulties can contribute to ineffective treatment, under-treatment, or lack of treatment, thereby needlessly prolonging medical conditions.

### BRIEF DESCRIPTION OF THE DRAWINGS

FIG. 1A illustrates a vial inserted in an example eyedrop dispenser;

FIG. 1B illustrates a vial removed from an example eyedrop dispenser;

FIG. 2 illustrates different views of the example dispenser;

FIG. 3 illustrates a collection of dispensers with different poka-yoke attachments;

FIG. 4 illustrates the collection of dispensers inserted into the corresponding poka-yoke-shaped receptacles in a container base station;

FIG. 5 illustrates an example dispenser communicating with the container base station and with a mobile device;

FIG. 6 illustrates example poka-yoke-distinguishing receptacles in the container base station;

FIG. 7 illustrates connectors and functionality of an example poka-yoke receptacle; and

FIG. 8 illustrates an example administrator interface for configuring dispensers;

FIG. 9A illustrates an example flow diagram for a patient retrieving a dispenser to apply an eyedrop and replacing the dispenser in the base station in its correct poka-yoke-distinguishing receptacle;

FIG. 9B illustrates an example flow diagram for the dispenser to apply an eyedrop;

FIG. 10 illustrates an example system embodiment; and

FIG. 11 illustrates an example flow diagram of communications flow from the home-based base stations to the database on the host server, to user charts, and to reports to care providers and users.

### DETAILED DESCRIPTION

A system, method, functionalities and computer-readable media are disclosed for allowing patients to properly self-administer eyedrops using spoken multi-lingual instructions and poka-yoke principles to minimize the potential for confusion, and by enabling communications between dispensers and a base station holding the dispensers and then to alerts and reports to users and their care givers. In this way, all authorized parties can view compliance and tracking data regarding how the eyedrops are administered to the patient.

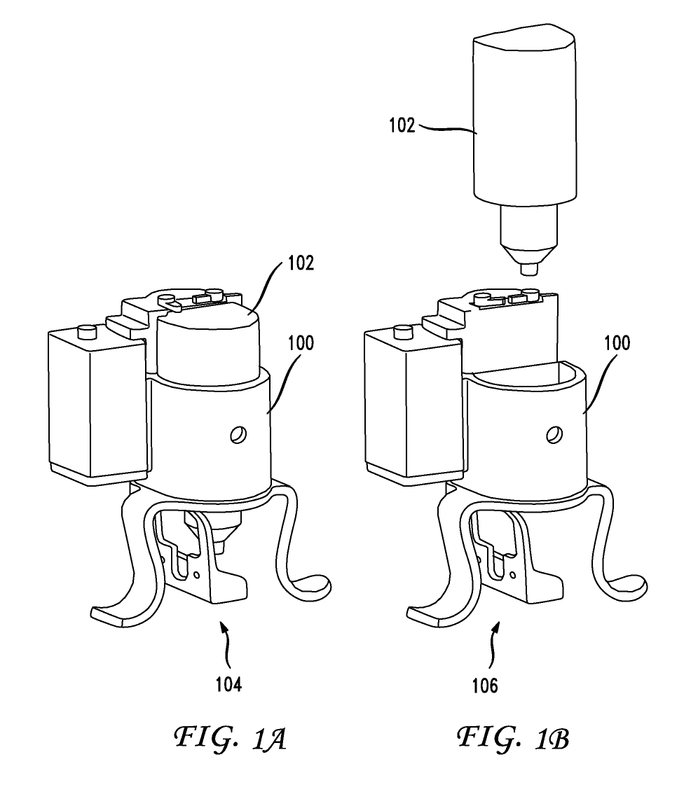

FIG. 1A illustrates an example dispenser 100 having a cavity in to which a vial of eyedrops 102 can be inserted, the dispenser 100 in a first configuration 104 with the vial of eyedrops 102 inserted into the dispenser 100. FIG. 1B illustrates a second configuration 106 with the vial of eyedrops 102 partially inserted into the dispenser 100. The dispenser 100 can hold the vial of eyedrops 102 in a compartment such as a customized chamber (e.g., cylinder), for example. The chamber can be any size or shape and can be sized for a specific vial type, or can be sized to interchangeably hold vials of multiple different sizes, such as 2.5, 5, 10, or 15 ml vials.

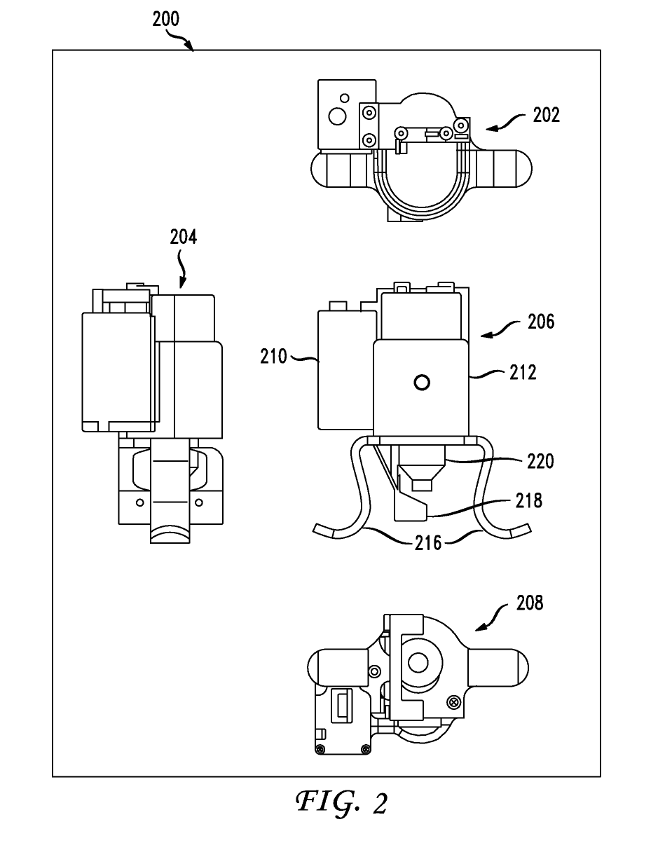

FIG. 2 illustrates a set 200 of different views of the example dispenser, including a top view 202, a first side view 204, a second side view 206, and a bottom view 208. The second side view shows some additional details of the design, including a side housing 210 that can contain components such as one or more of the motor, a cam mechanism, electrical components, a battery or power source, a processor, a connector, memory, wired or wireless data interfaces, an antenna, and so forth. The side housing 210 is illustrative, and can take a different shape, position, or size. The side housing 210 can be divided into several smaller housings in different locations on the dispenser. The side housing 210 can include a button 214 for activating a motor to apply pressure to squeeze only one single drop out of the vial nozzle 220. The motor, or electronics configured to control the motor, can activate the cam mechanism to apply one drop per press of the button 214, or can activate the motor to apply a number of drops specified by a prescription, for example. In one illustrative implementation, the motor is a 7 g sub-micro servo motor programmed to squeeze a single drop from the vial, having torque of 1.6 kg-cm or 22 oz-in, a speed of 0.12 sec/60″, and a size of 24×11×24 mm. In this example implementation, a Pic 12F microprocessor controls the servo motor, to stop and retract after a drop has been detected by a sensor 218, such as an LED infrared emitter and receiver.

A main housing 212 forms the cavity in to which a vial is inserted and secured. Spreaders 216 are attached at the bottom of the main housing 212, and are designed so, when placed over the eye, the eye lids of the patient are spread wide open for a large target, thereby preventing the user from blinking. The nozzle 220 of the vial inserted into the cavity extends through a hole in a bottom wall of the main housing 212 to administer the eyedrops to the patient's eye. Sensors 218 can be mounted on an arm for detecting that a drop has been expressed through the nozzle 220, among other metrics. For example, the sensors 218 can detect a size of the drop (such as via an LED infrared emitter and receiver), whether the drop impacted a surface of the eye (such as via a camera), whether the eye was open or closed at the point of impact, how many drops were expressed, an orientation of the dispenser (such as via an accelerometer or gyroscope), whether the spreaders 216 were in contact with the patient's face or eye lids, and so forth. The sensors 218 are illustrated as being mounted on the arm, but can be in different locations and can be distributed at multiple locations depending on their functionality and what the sensors 218 are designed to sense.

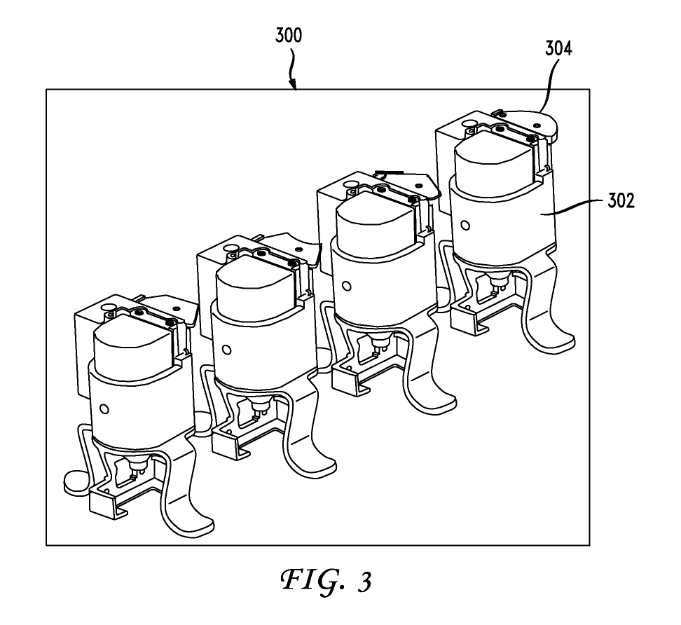

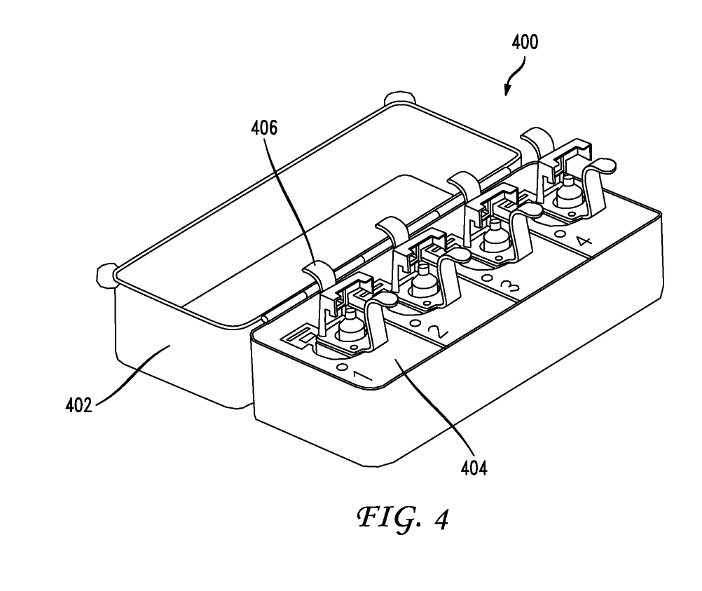

FIG. 3 illustrates a collection 300 of dispensers 302 with different poka-yoke attachments 304 specific to each compartment. In this example, the dispensers 302 are a standard size, and have different poka-yoke attachments affixed. The poka-yoke attachments may be removable and swappable between the dispensers 302, such as by an administrator or medical professional configuring the dispensers for a specific application or treatment regime. Alternatively, the poka-yoke attachments may be a permanently fixed part of the dispensers 302, such as part of a plastic head of the dispensers 302 molded at the time of manufacture. FIG. 4 illustrates a scenario 400 with the collection of dispensers 406 inserted into the corresponding poka-yoke receptacles 404 in a container base station 402. Each pair of a poka-yoke receptacle 404 and a corresponding dispenser 406 can be configured with matching shapes, heights, colors, textures, or materials, for example. The poka-yoke dispenser attachments are unique, so that each poka-yoke template fits in one and only one of the set of receptacles. The poka-yoke dispenser attachments can be color coded so that each one is a same unique color as its corresponding receptacle.

To further reinforce the pair, each of the receptacle 404 and a corresponding dispenser 406 can include matching identity marks, such as a letter, number, symbol, image, or combination thereof. In one example, the receptacle 404 and/or the corresponding dispenser 406 can include a pharmacy label showing the important information about the type of medicine, dosage information, and so forth. If the label is a display, e-Ink, or other dynamic graphical presentation, for example, the label can further present dynamically updated information, such as the remaining time to the next scheduled dose.

Instead of a printed paper label that shows medicine and dosage information, a base station and/or each corresponding dispenser can include other representations or ways to obtain that information. For example, in place of or in conjunction with a text label, visual indicators of the information can be printed thereon. In another example, a QR code is printed thereon that, if scanned by a QR code reader, can provide that information or link to a website that provides that information. In yet another example, a near-field communications (NFC) tag can be embedded in the base station or the dispenser to provide access to that information. In another example, a user can press an information button on either the base station face plate or the corresponding dispenser, and the system can play back an audio description of the information via an internal speaker or via a communicably connected audio playback device. The system can include a similar capability for video instructions. These examples are illustrative of various ways for representing additional information about a specific base station and its corresponding dispensers. In a similar way, the patient can access a treatment compliance history for the medicine associated with that dispenser, such as loading a secure, personalized treatment history web page. In yet another embodiment, the user can access performance information directly on his/her mobile device display with verbalized reports through the device's speakers.

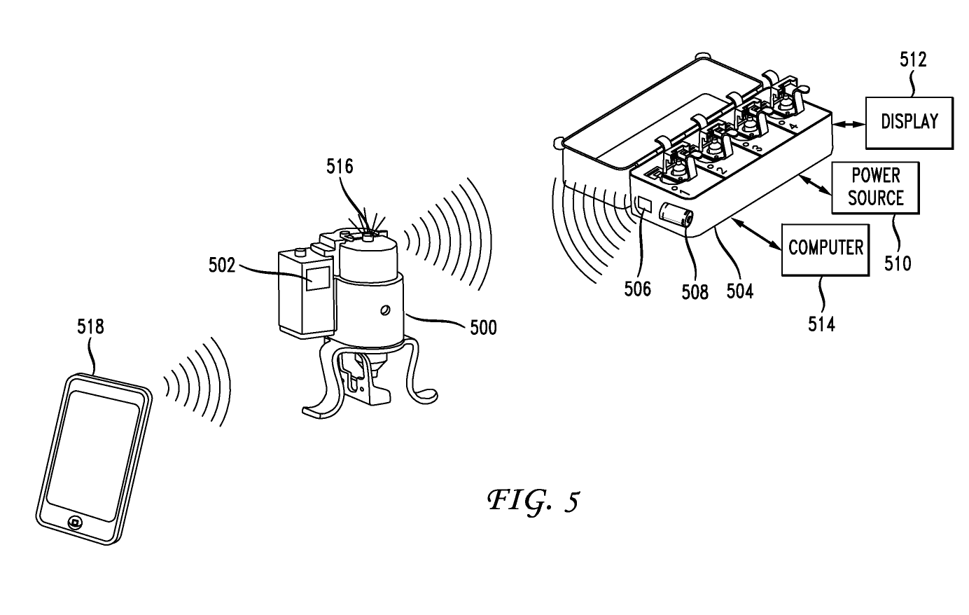

FIG. 5 illustrates an example dispenser 500 communicating with the container base station 504 and optionally with a mobile device 518. The dispenser 500 includes a communications module 502, which can include a processor, an antenna, memory, and other electrical and/or mechanical components, such as an LED indicator 516, a vibration module, or a display. The dispenser 500 can communicate with the base station 504, which has a corresponding communications module 506. The base station can optionally include components such as a display 512, a battery 508 or power source 510, a speaker, a motherboard, LEDs mounted to the faceplate representing each dispenser, sensors such as RFID or infrared, one or more communications modules, a button to ‘snooze’ or delay a cue to apply eye drops, voice chip or RAM chips that enable voiced instructions, power adapters for its embedded rechargeable batteries, and memory. Thus, it can communicate with an external computer 514 and with each of its paired dispensers. Either the base station 504 or the dispensers 500 can communicate with or be controlled by a smartphone 518 or other mobile device, such as via an application. While the signals depicted originate from the mobile device 518 to the dispenser 500, it should be understood that the communication protocol can permit unidirectional and bidirectional communications between the two units.

Assume the dispenser 500 starts in a receptacle in the base station 504 and the system is already configured according to a treatment regime prescribed by a doctor. At the appropriate time, multi-lingual announcements are spoken by the base station speakers to instruct the user to remove the dispenser due for dosing. The patient removes the proper dispenser 500 from the receptacle in the base station 504. If the user attempts to remove the wrong dispenser, the voiced announcement will instruct the user where to replace it and to extract the dispenser due for a dosing. The act of removing the dispenser 500 can trigger or activate portions of the dispenser 500, such as the sensors, the motor, or the communications module 506. These components are not needed while in the receptacle in the base station 504, and can thus be turned off to conserve power or placed in a lower-power consumption state.

After removing the dispenser 500, the patient positions the dispenser 500 over his or her eye such that the arms 216 press against the brow and cheekbone structure so as to prevent the eye from blinking. The user presses a button or otherwise activates the motor. The motor shaft causing the cam assembly to apply pressure to squeezes the vial to release only one drop of the medicine. Once the drop breaks the path of infrared sensors, or otherwise triggers a sensor, the dispenser 500 can illuminate the light 516, such as triggering a green LED for a few seconds. Alternatively, the dispenser 500 can trigger some other notification, such as a sound or an alert on the mobile device 518 or via the base station speaker 504. In this example, the dispenser 500 flashes an LED to confirm that a single drop has passed through the view of the infrared sensor placed in the base station, which detects that the LED is going off, and counts the drop as being successful. A second sensor can identify that the drop has contacted the eye and/or dispersed properly in the eye. The sensors can include infrared lights, RFID, cameras, microphones, lasers, radio sensors, and any other type of sensor that can be suitably incorporated within the dispenser physically, mechanically, and/or electrically. For example, the sensors can detect which eye the dispenser is applied to, and can ensure that the appropriate medication is only administered to the appropriate eye (i.e. left eye or right eye). The sensors can include a camera that detects facial or cranial features, which a processor uses to determine an orientation and position on the face based on those features. For example, a camera on the eyedropper can capture an image of an ear on one side and a nose on the other side. A processor can then determine orientation or directionality of the ear and nose to calculate over which eye the eyedropper is positioned. Similarly, the processor could determine the eye based on a position of a mouth and another eye. The sensors can even include high resolution imaging sensors that image blood vessels on the back of the eye to identify to which eye the eyedrops are applied. Additional sensors can ensure that only an authorized patient can administer the drops, such as via a biometric sensor that scans a retina or a fingerprint to confirm patient identity. Biometric identification sensors can also be incorporated on the base station 504, such as to unlock a locked receptacle so that the patient can retrieve the dispenser 500 therefrom.

The base station 504, as shown in FIG. 5, contains four receptacles that are uniquely shaped so that only one of a set of dispenser poke-yoke attachments fits therein. For example, if a doctor prescribes four different medications, then the base station 504 can include four unique receptacles, with each of such receptacle being shaped to receive a corresponding dispenser 500 with a matched poke-yoke disc. This assures that the dispenser can only be inserted into the correct receptacle, and the patient will not confuse the medications in each dispenser 500. Further, programming in the firmware of the base station microprocessor can trigger voiced announcements from the speaker that the wrong dispenser is being removed. The base station can include a processor or system-on-a-chip (SoC), such as an LPC1765 microchip. The base station 504 can include one or more surface LEDs or a pixel-based display 512 to alert which medication is to be taken. The base station 504 can provide directions on a display 512 regarding which eye, which medication, and at what time to apply the medication, or the base station 504 can provide such instructions to the mobile device 518 or a computer 514 via Bluetooth or other wired or wireless connection. For example, the base station 504 can contain an RN-42 chip to enable data transfer via Bluetooth to a MedSignals® hub device. The MedSignals® hub device can also provide cellular services for uploads to a computer 514 or server, and can also provide audio prompts, alerts, notifications, feedback, and reports. In one embodiment, such a MedSignals® device can control all or part of the functionality of both the base station 504 and the dispenser 500.

In one example, the dispenser 500 detects, via the sensors, that a successful application of the indicated eyedrop has occurred, and can not only light the LED 516 or provide other notification of success, but can also unlock the appropriate receptacle. The base station 504 can lock the receptacle or prevent reinsertion of the dispenser into the receptacle if the eyedrops are not applied. For example, the base station 504 can extend a metal rod or a plastic mesh into or over the receptacle to prevent re-insertion until the medicine has been applied to the patient's eye. The dispenser 500, the base station 504, the mobile device 518, or the computer 514 are examples of where the system can provide feedback when the dispenser 500 is successfully re-inserted into its corresponding receptacle in the base station 504. Examples of feedback for successful insertion can include displaying a green light, changing a color of a light from red to green, audible feedback such as a beep or playing an audio file, vibration, or locking the dispenser in place.

After a successful application of the medicine and reinsertion of the dispenser 500 into the base station, the receptacle can lock the dispenser in place until the next scheduled application, or some period of time prior to the next scheduled application. In a similar fashion, a controller or processer in the dispenser 500 can permit a specified number of drops to be expressed via the nozzle, and then prevent additional drops from being expressed until the next scheduled application time, until the dispenser has been returned to its receptacle in the base station, until a specified “timeout” duration has expired, or until some other condition has been satisfied. An administrator, doctor, or other authorized person or entity can override these lockouts, such as in the event that an eyedrop was expressed from the nozzle of the vial, but failed to reach the patient's eye and the patient wishes to attempt a second application.

The base station 504 can provide reminders via a speaker in the base station, SMS notifications, alerts to an application on the mobile device 518, blinking lights or LEDs, and so forth. An administrator or the user can establish rules for reminders, such as the timing, type, address, and so forth. In this way, the base station 504 can encourage the patient to apply the medication on the prescribed schedule.

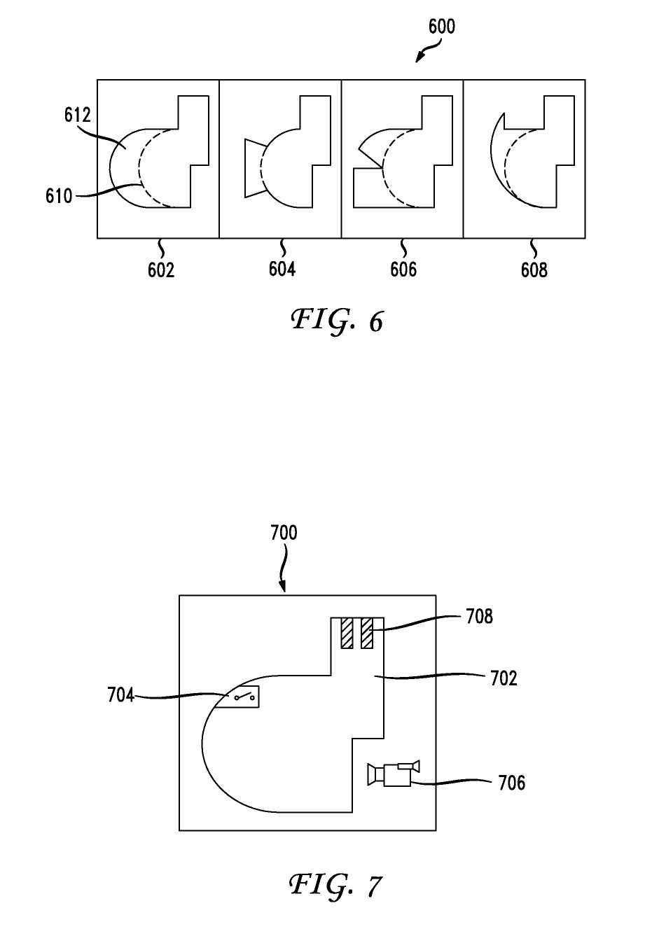

FIG. 6 illustrates an example 600 of poka-yoke receptacles 602, 604, 606, 608 in the container base station 504 of FIG. 5. Each receptacle has a shape patterned after a corresponding poke-yoke attachment to the dispenser. A standard ‘base’ shape shown by the dashed outline 610 matches an unmodified dispenser, and an additional poka-yoke shape 612 extends the base shape. In this example, poka-yoke extensions are affixed to the dispensers, so that each dispenser fits in one and only one of the receptacles 602, 604, 606, 608. The poka-yoke shapes are selected so that one of the dispensers cannot accidentally fit in to more than one receptacle. The receptacles can be modified to be different heights so that a desired portion of the appropriate dispenser, and only the appropriate dispenser, sits flush in the receptacle.

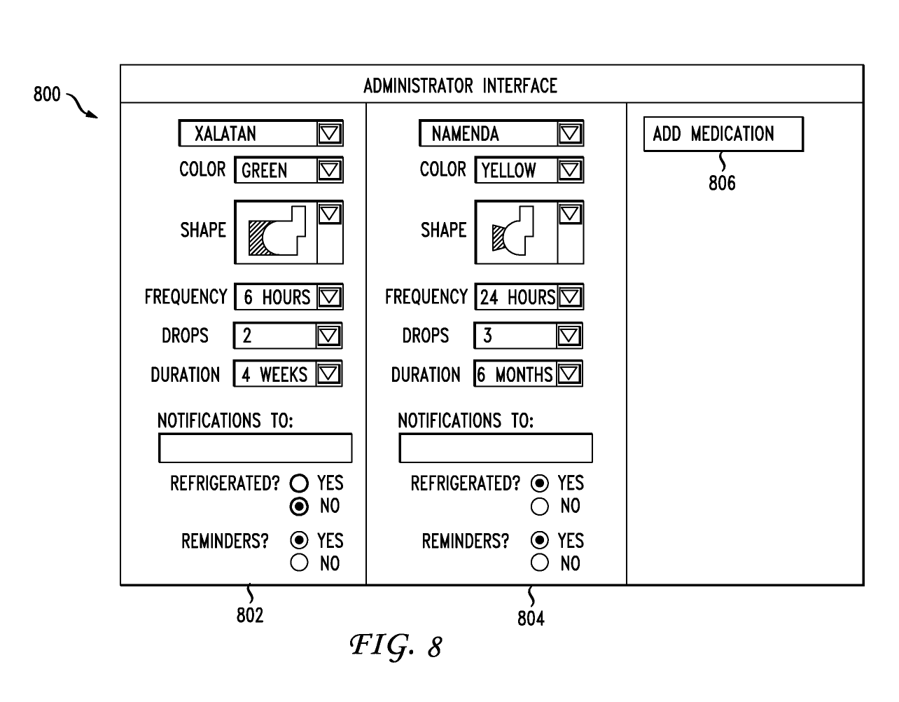

FIG. 7 illustrates a portion 700 of a base station showing connectors and functionality of an example poka-yoke receptacle 702. In this example, the base station can include one or more components to detect, control, and interface with the dispenser. For example, a receptacle 702 can include a micro-switch 704 to detect when the dispenser is removed and inserted. Similarly, the receptacle 702 can include a sensor to detect when the receptacle has been removed or inserted into a base station, if the receptacles 702 are replaceable. Replaceable receptacles can allow a doctor or administrator to easily insert or remove a desired number of receptacles in the base station when setting up the base station. Each receptacle can have its own processor and supporting electronic and mechanical elements, or can interface with a central processor and supporting electronic and mechanical elements via a system bus for the base station, for example. The receptacle 702 can include a sensor such as a light dependent resistor that will only count if the green LED on the dispenser is activated, indicating a successful application of the eyedrop to the patient's eye. A camera 706 can be embedded in, on, or under the receptacle 702 to visually inspect or verify the dispenser. For example, a camera can confirm that the proper dispenser was inserted. If the camera detects a different dispenser, the system can provide a visual or audible alert prompting the user to insert the dispenser into the correct receptacle. The camera can also be used to detect when the medicine is low or empty, if the sensors have been damaged or altered, and so forth. The receptacle 702 can further include electrical contacts 708 that make contact with the dispenser when the dispenser is inserted into the receptacle 702. The electrical contacts 708 can provide electrical power to charge or recharge a battery in the dispenser for powering the electronics, motor, sensors, and so forth. The base station can use the electrical contacts 708 to communicate with various components on the dispenser.

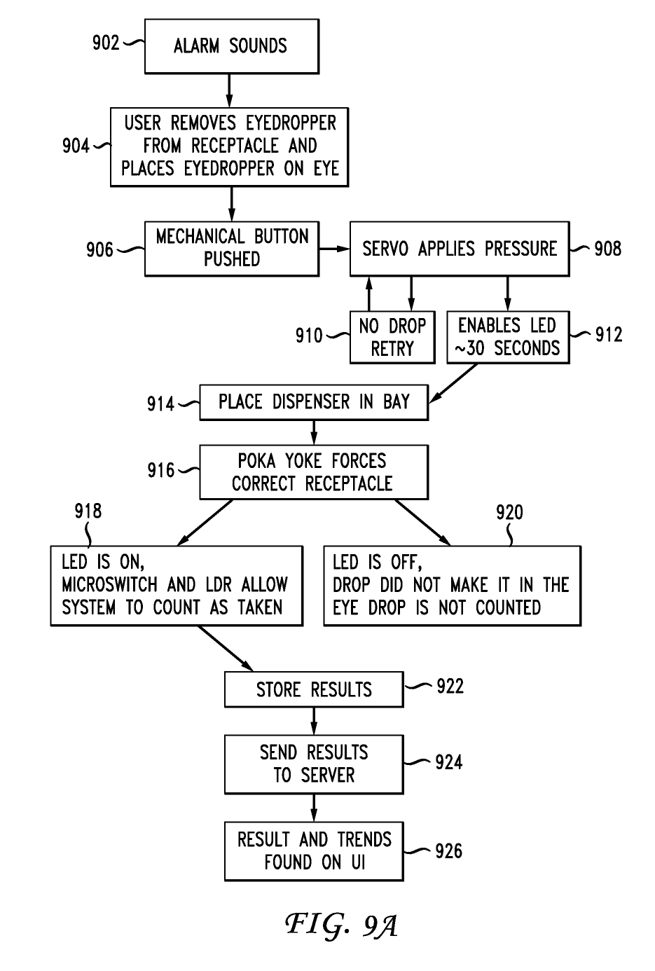

FIG. 8 illustrates an example administrator interface 800 for configuring dispensers. In this administrator interface, an administrator such as a care provider can establish settings and data for each receptacle and dispenser. This example interface 800 shows columns 802, 804, each column corresponding to a set of medicine, dispenser, and receptacle. The interface 800 allows the administrator to select the type of medicine, the color of the poka-yoke receptacle and dispenser, the poka-yoke shape, the frequency of applying the eyedrops, how many drops to apply at each interval, the duration of treatment, where and how to send notifications of compliance or non-compliance or other reports, whether the eyedrops must be refrigerated, whether and how to send reminders to the patient, and so forth. In the case of refrigerated medication, the base station can enable a temperature sensor with a trigger configured to sound an alarm or send a notification when the temperature rises above a certain threshold for more than a specified duration, according to the type of medication and its refrigeration requirements and/or tolerances.

The administrator can click an “Add Medication” button 806 to configure another receptacle, dispenser, and medication, and can likewise remove columns. In one embodiment, the administrator can access the interface via a web browser and to configure the base station. The interface 800 can provide lists of available options for each choice, such as the types of medications, available colors, available shapes, and so forth. The system can use a database of available poka-yoke shapes, and can automatically remove shapes that are too similar or that are compatible with already selected shapes, so that the administrator does not accidentally select receptacle shapes in to which more than one dispenser can be inserted. After the administrator configures the base station via the interface 800, the web server, computer, or other device can interface with the base station to apply the settings. In one embodiment, the base station itself hosts and serves the web interface. In another variation, a server provides the web interface and propagates those settings to the base station at the request of the administrator. While not shown, the system can provide similar corresponding interfaces for each dispenser, for each receptacle, for the base station, for alerts, for statistical reports, for compliance and security audits, and so forth. Each of these interfaces can provide live access to data recorded by the base station or dispensers.

A corresponding patient interface can allow the patient to configure permitted fields. For example, the patient may be able to change reminder settings, but may not be able to change the type of medication. The patient interface can show a history of each medication application, such as when the eyedrops were applied, when an application was skipped, and so forth. The user interface can be a website that displays the compliance of each patient. The administrator can configure who can access the patient interface, and may provide access to multiple groups and/or multiple patients.

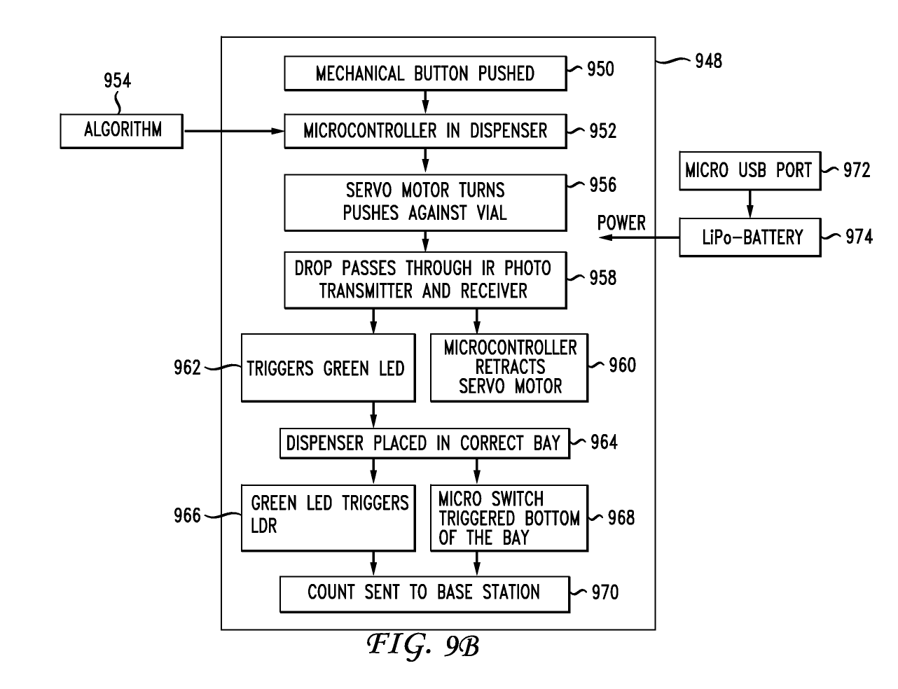

Having disclosed some basic system components and concepts, the disclosure now turns to the exemplary flow diagrams shown in FIGS. 9A and 9B. For the sake of clarity, the flow diagrams are described in terms of an exemplary system 1000 as shown in FIGS. 9A and 9B configured to practice all or some of the various steps. The steps outlined herein are examples and can be implemented in any combination thereof or in any order, including combinations that exclude, add, reorder, or modify certain steps.

FIG. 9A illustrates an example flow diagram for a patient retrieving a dispenser to apply an eye drop and replacing the dispenser in the base station. In this flow diagram, the system sounds an alarm 902 or provides some other reminder to the patient to apply the eyedrop. The patient removes the dispenser from the receptacle in the base station 904 and places the dispenser above his or her eye. Then the patient presses a mechanical button 906 on the dispenser, or provides some other input in lieu of pressing a mechanical button, such as issuing a voice command, or pressing a button on the base station or on a mobile device. Upon receiving this input, the servo applies pressure 908 to the vial in the dispenser. If the sensors do not detect a drop 910, the servo can retry. If the sensors detect a drop, the dispenser considers the operation a success, and can enable a success indicator 912, such as enabling an LED. The system can leave the LED enabled for a limited duration, such as 30 seconds, or until the dispenser is returned to the receptacle in the base station. Then the patient returns the dispenser to the receptacle 914. The poka-yoke design prevents the patient from returning the dispenser to an incorrect receptacle 916. Upon successful insertion of the dispenser into the appropriate receptacle, a camera in the receptacle detects the LED. If the LED is on, the system counts the eyedrop as successfully applied to the patient's eye. If the LED is off, the system does not count the eyedrop as successfully applied to the patient's eye. The system can store the results 922 of successful and/or unsuccessful eyedrop applications, then send those results to a server 924. Then a user such as a patient, a doctor, or an administrator can view the data as well as results and trends in the data 926.

FIG. 9B illustrates an example flow diagram for the dispenser 948 to apply an eyedrop. The user presses a mechanical button 950, for example. The microcontroller in the dispenser 952 handles the button press in accordance with a previously provided algorithm 954. The algorithm 954 can be generated based on information provided in the administrator interface 800, for example. The microcontroller causes the servo motor to turn and push against the vial 956. A sensor made up of an IR photo transmitter and receiver detects the drop passing through a predetermined location 958, and triggers a green LED 962 indicating success, and causes the microcontroller to retract the servo motor 960. Then, the user places the dispenser back in the correct receptacle 964. The LED triggers a light dependent resistor 966 and triggers a micro-switch in the receptacle 968, such as at the bottom or on a side wall of the receptacle. Then the system can send the count to the base station 970. The dispenser 948 can include a battery 974 that can be charged, for example, via a micro-USB port 972. Alternatively, the battery can be charged inductively from the base station.

If the base station is intended to remain in a refrigerator in order to keep specific medications at a desired low temperature, the base station can include a battery, or can be integrated into a power source within the refrigerator. For example, the base station can connect to a power source for a light socket in the refrigerator without interfering with the light's operation. Further, inasmuch as the base station is inside a refrigerator, any audio notifications may not be audible to the patient. Thus, a base station intended for storage in a refrigerator may provide an audio reminder to take the medication and also instruct a remote device to present a corresponding reminder. The base station can include a temperature sensor, and generate alerts or notifications to prompt the patient to return the base station to the refrigerator if left out for too long, or if the temperature drops below a desired threshold for more than a threshold amount of time.

While specific implementations are described herein, it should be understood that this is done for illustration purposes only. Other components and configurations may be used without parting from the spirit and scope of the disclosure.

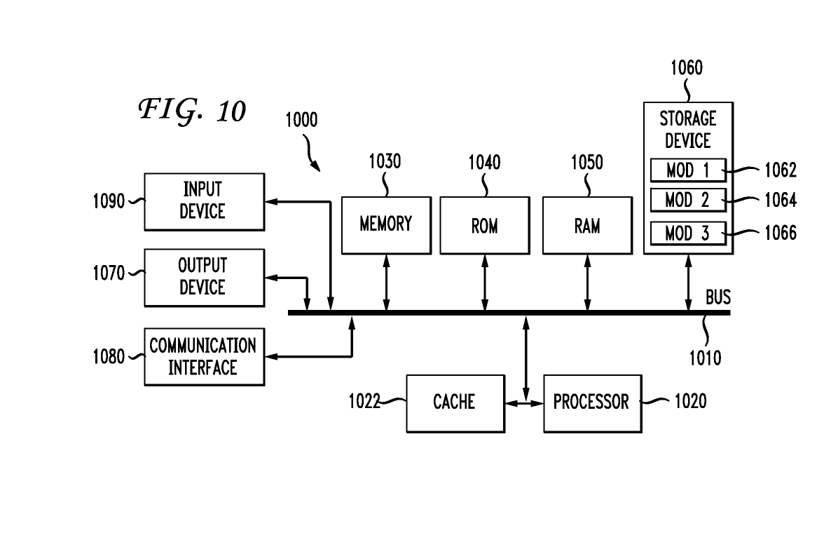

A brief description of a basic general purpose system or computing device in FIG. 10 which can be employed to practice the concepts is disclosed herein. With reference to FIG. 10, an exemplary system 1000 includes a general-purpose computing device 1000, including a processing unit (CPU or processor) 1020 and a system bus 1010 that couples various system components including the system memory 1030 such as read only memory (ROM) 1040 and random access memory (RAM) 1050 to the processor 1020. The system 1000 can include a cache 1022 of high speed memory connected directly with, in close proximity to, or integrated as part of the processor 1020. The system 1000 copies data from the memory 1030 and/or the storage device 1060 to the cache 1022 for quick access by the processor 1020. In this way, the cache provides a performance boost that avoids processor 1020 delays while waiting for data. These and other modules can control or be configured to control the processor 1020 to perform various actions. Other system memory 1030 may be available for use as well. The memory 1030 can include multiple different types of memory with different performance characteristics. It can be appreciated that the disclosure may operate on a computing device 1000 with more than one processor 1020 or on a group or cluster of computing devices networked together to provide greater processing capability. The processor 1020 can include any general purpose processor and a hardware module or software module, such as module 10 1062, module 2 1064, and module 3 1066 stored in storage device 1060, configured to control the processor 1020 as well as a special-purpose processor where software instructions are incorporated into the actual processor design. The processor 1020 may essentially be a completely self-contained computing system, containing multiple cores or processors, a bus, memory controller, cache, etc. A multi-core processor may be symmetric or asymmetric.

The system bus 1010 may be any of several types of bus structures including a memory bus or memory controller, a peripheral bus, and a local bus using any of a variety of bus architectures. A basic input/output (BIOS) stored in ROM 1040 or the like, may provide the basic routine that helps to transfer information between elements within the computing device 1000, such as during start-up. The computing device 1000 further includes storage devices 1060 such as a hard disk drive, a magnetic disk drive, an optical disk drive, tape drive or the like. The storage device 1060 can include software modules 1062, 1064, 1066 for controlling the processor 1020. Other hardware or software modules are contemplated. The storage device 1060 is connected to the system bus 1010 by a drive interface. The drives and the associated computer-readable storage media provide nonvolatile storage of computer-readable instructions, data structures, program modules and other data for the computing device 1000. In one aspect, a hardware module that performs a particular function includes the software component stored in a tangible computer-readable storage medium in connection with the necessary hardware components, such as the processor 1020, bus 1010, display 1070, and so forth, to carry out the function. In another aspect, the system can use a processor and computer-readable storage medium to store instructions which, when executed by the processor, cause the processor to perform a method or other specific actions. The basic components and appropriate variations are contemplated depending on the type of device, such as whether the device 1000 is a small, handheld computing device, a desktop computer, or a computer server.

Although the exemplary embodiment described herein employs the hard disk 1060, other types of computer-readable media which can store data that are accessible by a computer, such as magnetic cassettes, flash memory cards, digital versatile disks, cartridges, random access memories (RAMs) 1050, read only memory (ROM) 1040, a cable or wireless signal containing a bit stream and the like, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 1000, an input device 1090 represents any number of input mechanisms, such as a microphone for speech, a touch-sensitive screen for gesture or graphical input, keyboard, mouse, motion input, speech and so forth. An output device 1070 can also be one or more of a number of output mechanisms known to those of skill in the art. In some instances, multimodal systems enable a user to provide multiple types of input to communicate with the computing device 1000. The communications interface 1080 generally governs and manages the user input and system output. There is no restriction on operating on any particular hardware arrangement and therefore the basic features here may easily be substituted for improved hardware or firmware arrangements as they are developed.

For clarity of explanation, the illustrative system embodiment is presented as including individual functional blocks including functional blocks labeled as a “processor” or processor 1020. The functions these blocks represent may be provided through the use of either shared or dedicated hardware, including, but not limited to, hardware capable of executing software and hardware, such as a processor 1020, that is purpose-built to operate as an equivalent to software executing on a general purpose processor. For example the functions of one or more processors presented in FIG. 10 may be provided by a single shared processor or multiple processors. (Use of the term “processor” should not be construed to refer exclusively to hardware capable of executing software.) Illustrative embodiments may include microprocessor and/or digital signal processor (DSP) hardware, read-only memory (ROM) 1040 for storing software performing the operations described below, and random access memory (RAM) 1050 for storing results. Very large scale integration (VLSI) hardware embodiments, as well as custom VLSI circuitry in combination with a general purpose DSP circuit, may also be provided.

The logical operations of the various embodiments are implemented as: (1 ) a sequence of computer implemented steps, operations, or procedures running on a programmable circuit within a general use computer, (2) a sequence of computer implemented steps, operations, or procedures running on a specific-use programmable circuit; and/or (3) interconnected machine modules or program engines within the programmable circuits. The system 1000 shown in FIG. 10 can practice all or part of the recited methods, can be a part of the recited systems, and/or can operate according to instructions in the recited tangible computer-readable storage media. Such logical operations can be implemented as modules configured to control the processor 1020 to perform particular functions according to the programming of the module. For example, FIG. 10 illustrates three modules, namely, Mod1 1062, Mod2 1064 and Mod3 1066, which are modules configured to control the processor 1020. These modules may be stored on the storage device 1060 and loaded into RAM 1050 or memory 1030 at runtime or may be stored in other computer-readable memory locations.

Embodiments within the scope of the present disclosure may also include tangible and/or non-transitory computer-readable storage media for carrying or having computer-executable instructions or data structures stored thereon. Such tangible computer-readable storage media can be any available media that can be accessed by a general purpose or special purpose computer, including the functional design of any special purpose processor as described above. By way of example, and not limitation, such tangible computer-readable media can include RAM, ROM, EEPROM, CD-ROM or other optical disk storage, magnetic disk storage or other magnetic storage devices, or any other medium which can be used to carry or store desired program code means in the form of computer-executable instructions, data structures, or processor chip design. When information is transferred or provided over a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable media.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

Other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

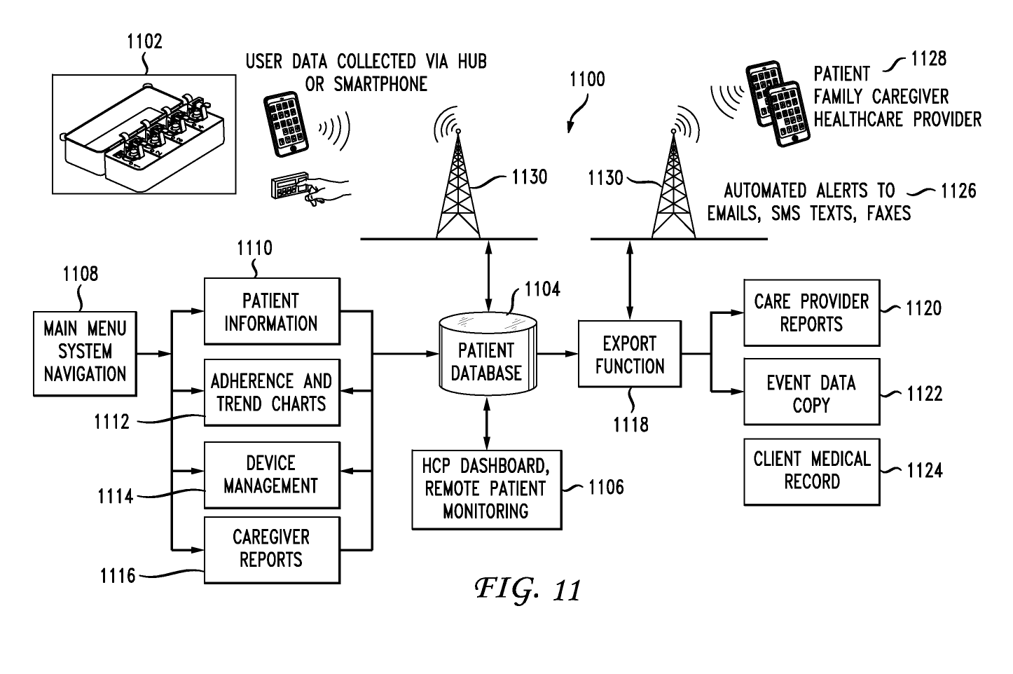

FIG. 11 illustrates an example flow diagram 1100 of communications flow from the home-based base stations 1102 to a patient database 1104 on a host server, to user charts, and to reports to care providers and users 1128. In this example, the base station 1102 can communicate with handheld or mobile devices as well as with a server over the Internet, for example. The server communicates with a network interface 1130. The network interface 1130 can be a wired or wireless network interface. The data received from the base station 1102 is stored in a patient database 1104 and made available for incorporation or presentation via an dashboard for remote patient monitoring 1106, or via a menu system 1108 that has sections such as a patient information page 1110, adherence and trend charts 1112, device management 1114, and caregiver reports 1116. Some of these sections may simply present data from the patient database 1104, while others, such as device management 1114, may allow a user to manage or communicate with the base station 1102.

The patient database 1102 can include an export function 1118 for providing data via external interfaces, such as a care provider report 1120, a client medical record 1124, or to enable event data from the base station 1102 to be copied 1122 or saved. A notifier can analyze the patient database 1104 to determine when all or part of the data in the database should be exported 1118 to provide automated alerts 1126 to specific individuals or groups, such as a patient, family caregiver, or healthcare provider. The rules for generating these automated alerts may be different for each individual or group. For example, a family caregiver may have a separate rule set from a healthcare provider. Further, the rules can dictate the content and delivery approach for the notifications, whether by telephone call, SMS, instant messaging, fax, email, postal mail, and so forth. The notifications can be text-based, but can include any other form of data from the patient database 1104, such as images, video, audio, statistical usage data in chart form, and so forth. The export function can include a data processor that prepares the data for presentation, or makes the data available via an API to authorized entities, in accordance with safety and privacy compliance standards.

The various embodiments described above are provided by way of illustration only and should not be construed to limit the scope of the disclosure. Various modifications and changes may be made to the principles described herein without following the example embodiments and applications illustrated and described herein, and without departing from the spirit and scope of the disclosure.

We claim:

1. An dispenser base station comprising:
a plurality of dispenser receptacles, each receptacle being configured to receive and contain a respective specifically shaped dispenser from a plurality of dispensers associated with the dispenser base station, wherein each of the plurality of dispensers is uniquely shaped and is configured to receive an eyedrop medicine container;
a dispenser communications interface configured to receive eyedrop usage data from at least one of the plurality of dispensers;
a storage device configured to store the eyedrop usage data; and
an external communications interface configured to transmit the eyedrop usage data to at least one external device.

2. The dispenser base station of claim 1, wherein each dispenser receptacle is uniquely colored to match its respective specifically shaped dispenser.

3. The dispenser base station of claim 1, wherein each dispenser receptacle is uniquely labeled to match its respective specifically shaped dispenser.

4. The dispenser base station of claim 1, further comprising:
a configuration interface for receiving configuration data for the plurality of dispensers or receptacles; and
a programming interface for programming each of the plurality of dispensers according to the configuration data.

5. The dispenser base station of claim 1, wherein the external communications interface comprises a web server for providing the eyedrop usage data as web pages.

6. The dispenser base station of claim 1, wherein the plurality of dispenser receptacles are removable from the dispenser base station.

7. The dispenser base station of claim 1, further comprising a processor configured to control at least one of the dispenser communications interface, the storage device, and the external communications interface.

8. The dispenser base station of claim 1, wherein the external communications interface transmits data to an application on a mobile device via an application programming interface (API).

9. The dispenser base station of claim 1, further comprising a power source.

10. The dispenser base station of claim 9, wherein the power source comprises at least one of a battery, a wired connection to an external power supply, and an inductive charging receiver.

11. An dispenser comprising:
a chamber for holding an eyedrop medicine container, wherein the eyedrop medicine container is removable from the chamber, and wherein the eyedrop medicine container is held in place in the chamber via at least one restraint;
a micro-motor configured to apply pressure to the eyedrop medicine container while in the chamber to release an indicated number of drops of medicine from the eyedrop medicine container;
a first sensor configured to gather first data describing drops of medicine from the eyedrop medicine container;
a second sensor configured to detect second data describing how the drops of medicine contacts a surface of an eye;
a notification module for generating a notification based on at least one of the first data and the second data;
a communication interface for transmitting at least one of the first data and the second data to a dispenser base station;
a switch which, when triggered, causes at least one of the first data and the second data to be reset; and
a poka-yoke portion configured to allow the dispenser to enter one and only one receptacle in the base station while preventing the dispenser to enter other receptacles in the base station.

12. The dispenser of claim 11, wherein the chamber is adjustable to hold different types and shapes of eye-drop medicine containers.

13. The dispenser of claim 11, wherein the communication interface can further transmit at least one of the first data and the second data to a remote computing device via a communications network.

14. The dispenser of claim 11, wherein the communication interface can receive instructions for controlling, via a processor, at least one of the micro-motor, the first sensor, the second sensor, the notification module, and the switch.

15. The dispenser of claim 11, wherein the communication interface communicates with a remote device to provide, via the remote device, at least one of an audible notification, a visual notification, a vibration-based notification, a text-based notification, an alarm, transmission to an external device, and a log entry.

16. The dispenser of claim 11, wherein the poka-yoke portion is colored to match a corresponding receptacle in the base station.

17. The dispenser of claim 11, further comprising:
a locking mechanism that prevents the micro-motor from applying pressure to the eyedrop medicine container;
a controller for the locking mechanism that locks and unlocks the locking mechanism.

18. The dispenser of claim 17, wherein the controller locks and unlocks the locking mechanism based on at least one of timing, whether the dispenser is in the base station, whether the eyedrop medicine container is inserted into the cavity, confirmation of a patient identity, an amount of medicine in the eyedrop medicine container, and whether the first data and the second data have been reported to the base station.

19. The dispenser of claim 11, wherein the notification module comprises an LED which is illuminated upon successful application of medicine from the eyedrop medicine container, as detected by one of the first sensor or second sensor, until a timer expires or until the dispenser is returned to the base station.

20. The dispenser of claim 11, further comprising a temperature sensor, wherein if the temperature sensor detects a temperature above a temperature threshold for more than a threshold amount of time, the notification module generates a temperature alert, and wherein the temperature threshold and the threshold amount of time are based on medicine in the eyedrop medicine container.
